(19) United States
(12) Patent Application Publication
(10) Pub. No.: US 2026/0066059 A1
(43) Pub. Date: Mar. 5, 2026

(54) METHOD AND APPARATUS FOR DETERMINING SYNTHESIS RECIPE USING NEURAL NETWORK MODEL AND TRAINING METHOD OF NEURAL NETWORK MODEL

(71) Applicants: SAMSUNG ELECTRONICS CO., LTD., Suwon-si (KR); RESEARCH & BUSINESS FOUNDATION SUNGKYUNKWAN UNIVERSITY, Suwon-si (KR)

(72) Inventors: Youngchun KWON, Suwon-si (KR); Seokho KANG, Gwacheon-si (KR); Joonhyuk CHOI, Suwon-si (KR)

(73) Assignees: SAMSUNG ELECTRONICS CO., LTD., Suwon-si (KR); RESEARCH & BUSINESS FOUNDATION SUNGKYUNKWAN UNIVERSITY, Suwon-si (KR)

(21) Appl. No.: 19/319,252

(22) Filed: Sep. 4, 2025

(30) Foreign Application Priority Data
Sep. 4, 2024 (KR) 10-2024-0120274
Oct. 24, 2024 (KR) 10-2024-0146908

(51) Int. Cl. G16C 20/40; G06N 3/045; G16C 20/10; G16C 20/70; G16C 20/80; G16C 20/90

(52) U.S. Cl. CPC G16C 20/40; G06N 3/045; G16C 20/10; G16C 20/70; G16C 20/80; G16C 20/90

(57) ABSTRACT

A method performed by at least one processor, includes receiving a query comprising at least one of a product, a reactant, a reagent, and a reaction condition; generating a query embedding vector corresponding to the query by inputting the query into a neural network model; extracting a candidate embedding vector from among a plurality of embedding vectors based on a similarity between the query embedding vector and the plurality of embedding vectors corresponding to reaction records stored in a database; and outputting a synthesis recipe corresponding to the query by retrieving a candidate reaction record corresponding to the candidate embedding vector in the database.

### CROSS-REFERENCE TO RELATED APPLICATIONS

This application claims priority under 35 USC § 119(a) to Korean Patent Application No. 10-2024-0120274, filed on Sep. 4, 2024, and Korean Patent Application No. 10-2024-0146908, filed on Oct. 24, 2024, in the Korean Intellectual Property Office, the entire disclosures of which are incorporated by reference herein for all purposes.

### BACKGROUND

### 1. Field

The following embodiments relate to a method and apparatus for predicting a synthesis recipe using a neural network model and a learning method of the neural network model.

### 2. Description of Related Art

A method in which a series of processes (e.g., data search, experiment design, experiment preparation, and the like) performed during synthesis of organic materials to discover new materials are performed by various neural network models and/or artificial intelligence (AI) algorithms is being considered. Researchers may conduct experiments by determining materials (e.g., starting materials and reactants) needed to make an experimental product and designing a synthetic scheme (e.g., a catalyst, solvent, temperature, and the like). The development of new materials and/or new drugs may require a very high amount of time and cost, and various experimental environments or experimental conditions may bias accumulated data.

AI algorithms may not be able to easily find new synthesis methods for novel molecules, as the AI algorithms may only display a subset of synthesis recipe items for experiments or simply search for similar experimental papers. In addition, when any of the items included in the synthesis recipe are missing, the experiment may be conducted based on the knowledge of a researcher, making it difficult to conduct an objective experiment because the experimental results may depend on the background knowledge of the researcher.

The above description has been possessed or acquired by the inventor(s) in the course of conceiving the present disclosure and is not necessarily an art publicly known before the present application is filed.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

According to an aspect of the disclosure, a method performed by at least one processor, the method including receiving a query including at least one of a product, a reactant, a reagent, and a reaction condition; generating a query embedding vector corresponding to the query by inputting the query into a neural network model; extracting a candidate embedding vector from among a plurality of embedding vectors based on a similarity between the query embedding vector and the plurality of embedding vectors corresponding to reaction records stored in a database; and outputting a synthesis recipe corresponding to the query by retrieving a candidate reaction record corresponding to the candidate embedding vector in the database.

The neural network model may be trained to: generate a target vector corresponding to the product for each synthesis recipe and a prediction vector corresponding to at least one of the reactant, the reagent, and the reaction condition, and determine the synthesis recipe corresponding to the query or an embedding vector of the synthesis recipe corresponding to the query based on the target vector and the prediction vector.

The extracting the candidate embedding vector may include: determining a similarity score based on a Euclidean distance between the query embedding vector and the plurality of embedding vectors; and extracting the candidate embedding vector from among the plurality of embedding vectors based on the similarity score.

The method may include reducing a dimension of the query using principal component analysis (PCA), in which the inputting the query to the neural network model further includes: inputting the query of which the dimension is reduced to the neural network model to generate the query embedding vector.

The product, the reactant, and the reagent may have a molecular graph form that represents a three-dimensional (3D) molecular structure in a form of a graph.

The method may further include generating a visual of one or more synthesis recipes corresponding to the query by displaying the candidate reaction record corresponding to the candidate embedding vector on an organic synthesis map.

The method may further include generating, in a form of a graph, a 3D molecular structure to represent at least one element among the product, the reactant, and the reagent included in the query.

The method may further include updating the neural network model based on user feedback on the synthesis recipe; and inputting the query into the updated neural network model to generate an updated query embedding vector.

The updating of the neural network model may further include: storing an evaluation score corresponding to the user feedback in a query database; updating the reaction records by reflecting the evaluation score in a similarity of an embedding vector corresponding to a corresponding synthesis recipe in the query database; and updating the neural network model based on the updated reaction records.

According to an aspect of the disclosure, a training method of a neural network model performed by at least one processor for determining a synthesis recipe, includes receiving a training query including at least one of a product, a reactant, a reagent and a reaction condition; generating a target vector corresponding to the product for the synthesis recipe and a prediction vector corresponding to at least one of the reactant, the reagent, and the reaction condition by inputting the training query into the neural network model; and training, based on the target vector and the prediction vector, the neural network model to determine the synthesis recipe corresponding to the training query or an embedding vector of the synthesis recipe corresponding to the training query.

The neural network model may include: at least one graph neural network (GNN) including an encoder configured to extract a representation vector of a molecular unit corresponding to each of the product, the reactant, and the reagent; a plurality of projection heads configured to convert the representation vector of the molecular unit corresponding to each of the product, the reactant, and the reagent to a low-dimensional latent vector; and a feed-forward neural network (FNN) configured to output a reaction vector corresponding to the reaction condition.

The plurality of projection heads may include at least one of: a first projection head configured to convert a first representation vector corresponding to the product to a first latent vector; a second projection head configured to convert a second representation vector corresponding to the reactant to a second latent vector; and a third projection head configured to convert a third representation vector corresponding to the reagent to a third latent vector.

The generating the target vector and the prediction vector may further include: applying the first representation vector to the first projection head to generate the first latent vector corresponding to the product to be the target vector; applying the second representation vector to the second projection head to determine the second latent vector corresponding to the reactant; applying the third representation vector to the third projection head to determine the third latent vector corresponding to the reagent; applying the reaction condition to the FNN to extract a reaction vector corresponding to the reaction condition; and generating the prediction vector based on at least one of the second latent vector, the third latent vector, and the reaction vector.

The at least one GNN may be configured to, in response to a missing element existing among the product, the reactant, and the reagent, provide a distribution value of a neighboring reaction record adjacent to a reaction record corresponding to the missing element in a database as a value corresponding to the missing element.

The plurality of projection heads may be configured to, in response to a missing element existing among the product, the reactant, and the reagent input to the encoder, output a zero vector corresponding to the missing element.

The training of the neural network model may include training the neural network model by contrastive learning based on the target vector and the prediction vector.

The training of the neural network model by contrastive learning may include: configuring a positive pair by the target vector and the prediction vector corresponding to a same synthesis recipe; configuring a negative pair by the target vector and the prediction vector corresponding to different synthesis recipes; and training the neural network model to learn a representation that causes the positive pair come closer to each other and the negative pair move away from each other in a vector space.

The method may further include receiving user feedback on the synthesis recipe generated by the neural network model; updating a database based on additional training data generated by the user feedback; and updating the neural network model based on the updated database.

According to an aspect of the disclosure, a non-transitory computer-readable storage medium having instructions stored therein, which when executed by a processor cause the processor to execute a method including: receiving a query including at least one of a product, a reactant, a reagent, and a reaction condition; generating a query embedding vector corresponding to the query by inputting the query into a neural network model; extracting a candidate embedding vector from among a plurality of embedding vectors based on a similarity between the query embedding vector and the plurality of embedding vectors corresponding to reaction records stored in a database; and outputting a synthesis recipe corresponding to the query by retrieving a candidate reaction record corresponding to the candidate embedding vector in the database.

According to an aspect of the disclosure, an apparatus includes: a communication interface configured to receive a query including at least one of a product, a reactant, a reagent, and a reaction condition; a memory configured to store one or more instructions and a neural network model; and a processor operatively coupled to the memory, in which the one or more instructions, when executed by the processor, cause the apparatus to: generate a query embedding vector corresponding to the query by inputting the query to the neural network model, extract a candidate embedding vector from among a plurality of embedding vectors based on a similarity between the query embedding vector and the plurality of embedding vectors corresponding to reaction records stored in a database, and output a synthesis recipe corresponding to the query by retrieving a candidate reaction record corresponding to the candidate embedding vector in the database.

Other features and aspects will be apparent from the following detailed description, the drawings, and the claims.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a flowchart of a method of predicting a synthesis recipe according to one or more embodiments;

FIG. 2 illustrates a structure and an operation of a neural network model according to one or more embodiments;

FIG. 3A illustrates a process of updating a neural network model by reflecting user feedback according to one or more embodiments;

FIG. 3B illustrates a process of generating an organic synthesis map based on search results corresponding to a user query according to one or more embodiments;

FIG. 3C illustrates a method of extracting reaction records by similarity scores when a user query is given according to one or more embodiments;

FIG. 3D illustrates an example of candidate embedding vectors stored in a query database according to one or more embodiments;

FIG. 4 illustrates a visualized result of searching for synthesis recipes corresponding to a query according to one or more embodiments;

FIG. 5 is a flowchart of a method of predicting a synthesis recipe according to one or more embodiments;

FIG. 6 illustrates search results after a neural network model is updated based on user feedback according to one or more embodiments;

FIG. 7 illustrates a process in which user feedback is reflected in prediction results of a neural network model according to one or more embodiments;

FIG. 8 is a flowchart of a learning method of a neural network model according to one or more embodiments; and

FIG. 9 is a block diagram of an apparatus for predicting a synthesis recipe according to one or more embodiments.

### DETAILED DESCRIPTION

The following detailed structural or functional description is provided as an example only and various alterations and modifications may be made to the embodiments. Accordingly, the embodiments are not construed as limited to the disclosure and should be understood to include all changes, equivalents, and replacements within the idea and the technical scope of the disclosure.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

Although terms, such as first, second, and the like are used to describe various components, the components are not limited to the terms. These terms should be used only to distinguish one component from another component. For example, a first component may be referred to as a second component, and similarly the second component may also be referred to as the first component.

It should be noted that if it is described that one component is “connected”, “coupled”, or “joined” to another component, a third component may be “connected”, “coupled”, and “joined” between the first and second components, although the first component may be directly connected, coupled, or joined to the second component.

The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

As used herein, expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, or all of a, b, and c.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, embodiments will be described in detail with reference to the accompanying drawings. When describing the embodiments with reference to the accompanying drawings, like reference numerals refer to like elements and a repeated description related thereto will be omitted.

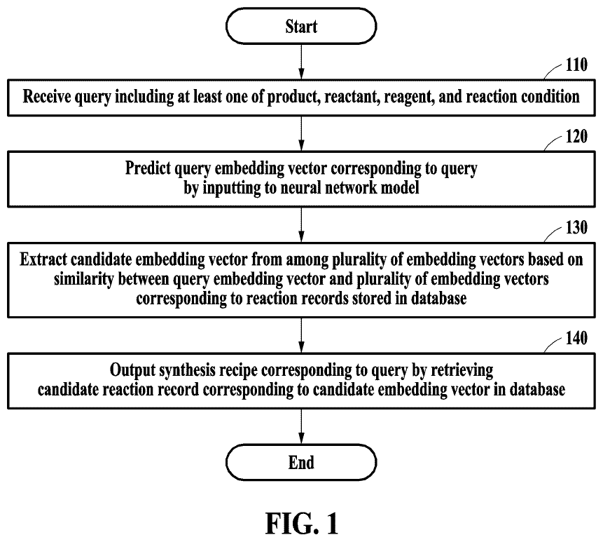

FIG. 1 is a flowchart of a method of predicting a synthesis recipe according to one or more embodiments. In the following embodiments, operations may be performed sequentially, but are not necessarily performed sequentially. For example, the order of the operations may be changed and at least two of the operations may be performed in parallel. Furthermore, when two or more operations are performed in parallel, the operations may be started at different times and/or end at different times.

Referring to FIG. 1, an apparatus (hereinafter, “prediction apparatus”) for predicting synthesis recipes according to one or more embodiments may output (e.g., visualize) the synthesis recipes predicted through operations 110 to 140.

The prediction apparatus may be implemented as various types of devices such as, for example, a personal computer (PC), a server device, a mobile device, an embedded device and the like, and more specifically, for example, as a smartphone, a tablet device, an augmented reality (AR) device, an Internet of Things (IoT) device and/or a medical device, which performs voice recognition, image recognition, and image classification based on a neural network, but examples are not limited thereto. Furthermore, the prediction apparatus may be a dedicated hardware (HW) accelerator installed in the above-described devices, or may be an HW accelerator such as a neural processing unit (NPU), a tensor processing unit (TPU), a neural engine, and the like, which are dedicated modules for operating a neural network, but is not limited thereto.

In operation 110, the prediction apparatus may receive a query including at least one of a product, a reactant, a reagent, and a reaction condition. In one or more examples, the query may be a text input describing the product, reactant, reagent, or the reaction condition. In one or more examples, the query may be one or more images of the product, reactant, reagent, or the reaction condition. In one or more examples, query may include both text and/or one or more images. The “product” may refer to a target material (e.g., a compound) to be produced by a synthesis recipe. The “reactant” may refer to material(s) used to produce a product by a chemical reaction according to the synthesis recipe. The “chemical reaction” may refer to a process in which a reactant undergoes a chemical transformation to produce a product under specific reaction conditions including a chemical context (e.g., a reagent, catalyst, solvents) and operating conditions (e.g., temperature and pressure). For example, the chemical reaction may be a process in which any chemical material changes into another material through a chemical change or chemical reaction. In one or more examples, the material that has been changed (produced) through the chemical reaction may correspond to the product, and the material before changing in the chemical reaction may correspond to the reactant. Chemical reactions may occur from a variety of reaction modes. A “reaction mode” may correspond to a chemical reaction scheme for producing a product using reactants being synthesized. The reaction mode may include, the Suzuki-Miyaura reaction mode, the Buchwald reaction mode, and/or the arylation reaction mode, but is not necessarily limited thereto. A plurality of reaction modes may be provided, for example, depending on structure information (e.g., A molecular structure+B molecular structure=C molecular structure) of the reactants and structure information of the products. In one or more examples, “structure information” may refer to the structure of a material at an atomic level. Although the specification discloses the product as a compound, as understood by one of ordinary skill in the art, the product may be a single element that is modified according to the synthesis recipe to produce the product.

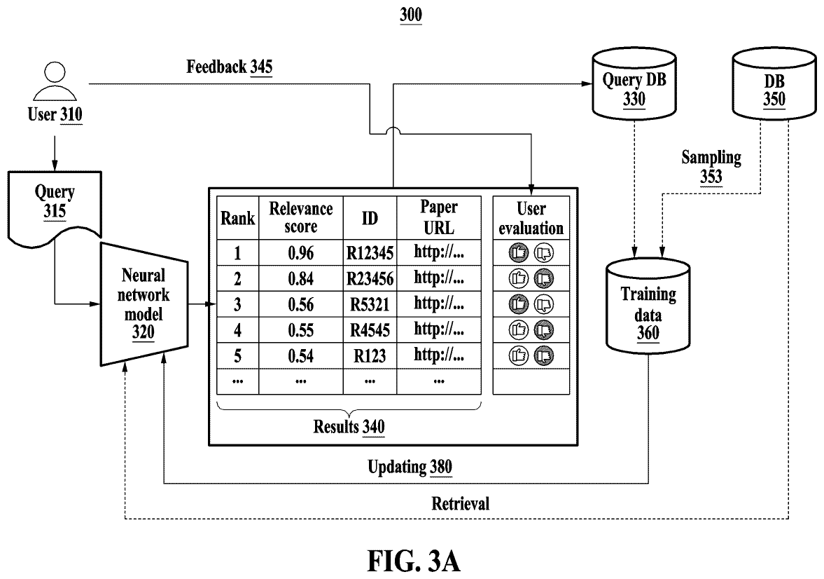

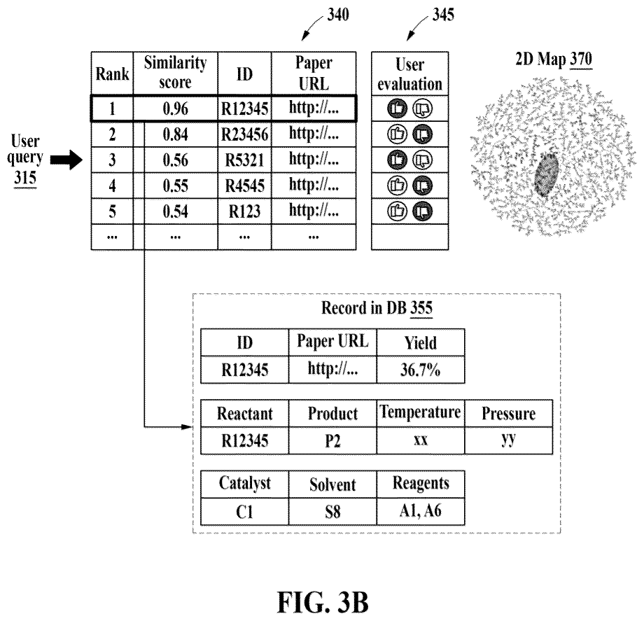

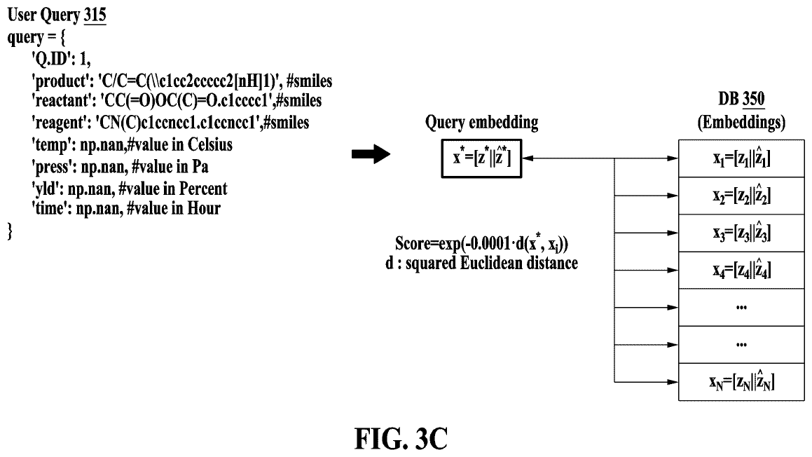

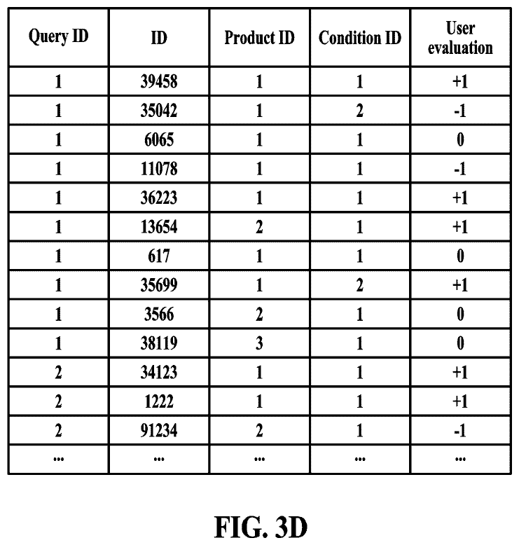

Identifying and optimizing chemical reactions is important in developing new functional materials. The prediction apparatus may obtain a synthesis recipe including information on a synthesis path of a target material to be synthesized by utilizing a chemical reaction database (e.g., a chemical reaction database 350 of FIG. 3A) as a resource. As will be described in more detail below, the chemical reaction database may include information on chemical reactions that have been experimentally verified and published in chemical literature. The chemical reaction database may be used to replicate and/or refine chemical reactions. The chemical reaction database may include, for example, Reaxys, Sci-finder, the United States Patent and Trademark Office (USPTO), and the Open Reaction Database (ORD), or any other suitable database known to one of ordinary skill in the art.

The “reagent” may refer to chemical agents used in a reaction for the detection or quantification of a material through a chemical experiment. For example, common chemicals used to make salt, ester, and other simple derivatives may be referred to as “reagents.” In addition to general reagents used for dissolution, precipitation, acidity adjustment, and various reactions, such as acid, alkali, and salt, the reagents may also include analytical reagents such as Nessler's reagent or Millon's reagent used for qualitative analysis and/or quantitative analysis by special reactions. For example, Nessler's reagent may be used to detect ammonia, and Millon's reagent may be used to detect the presence of soluble proteins and tyrosine The reagents may be divided into inorganic reagents, which are inorganic compounds, and organic reagents, which are organic compounds. The reagents may also be classified according to a state of the reagent, such as solid reagents, liquid reagents, and gaseous reagents. Additionally, the reagents may include catalysts, solvents, bases, and ligands.

The reaction conditions may be factors that affect a chemical reaction, including, but not necessarily limited to, temperature, pressure, density, humidity, reaction time, catalyst, ligand, bases, solvents, ratio, and/or yield.

In addition to the products, reactants, reagents, and reaction conditions described above, a query may further include user-specified search criteria, search preferences of the user, and requirements of the user. The prediction apparatus may retrieve relevant records or relevant information from the chemical reaction database based on search criteria included in a query. For example, when the query includes a product, the prediction apparatus may retrieve and/or predict a synthesis recipe including reactants, reagents, and reaction conditions for producing the product. When the query includes reactants and reaction conditions (or reactants, reagents, and reaction conditions), the prediction apparatus may retrieve and/or predict various synthesis recipes that may synthesize a product that may be produced (synthesized) by the reactants and reaction conditions (or reactants, reagents, and reaction conditions). When the query includes all of products, reactants, reagents, and reaction conditions, the prediction apparatus may retrieve and/or predict synthesis recipes or related papers including various reaction schemes or various synthesis paths from reactants to products. The query may further be associated with a user ID, where previous queries associated with the user ID may be referenced when predicting a synthesis recipe.

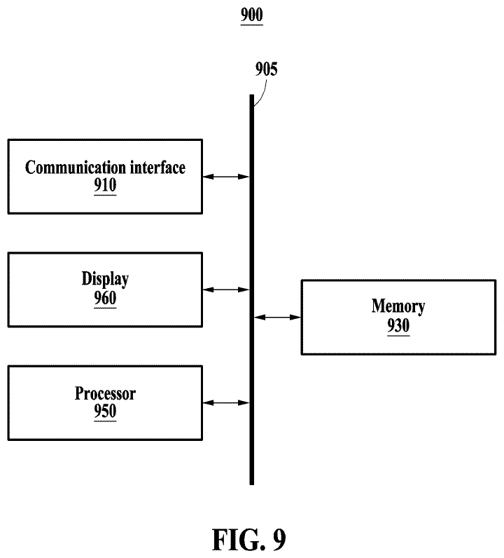

The prediction apparatus may predict, retrieve, or generate a synthesis recipe that reflects the search preferences and requirements of a user. A query may be input, for example, through a display screen of the prediction apparatus by a user interface (UI), or may be received from another device (e.g., a user terminal or a server) through a communication interface (e.g., a communication interface 910 of FIG. 9).

In one or more examples, the products, reactants, and reagents included in the query may have a molecular graph form that represents a three-dimensional (3D) molecular structure in a graph form but are not necessarily limited thereto. The reactants may include molecular graphs representing a plurality of reactant molecules corresponding to different chemical reactions. The product may include a single molecule graph representing a product molecule.

For example, an arbitrary molecule may be expressed by an undirected graph G=(V, E). The parameter V may denote a set of nodes associated with heavy atoms within a molecule. The parameter E may denote a set of edges associated with chemical bonds between heavy atoms.

Each of the molecular graphs and the single molecule graph may include node vectors representing node features corresponding to heavy atoms within the molecule, and edge vectors representing edge features corresponding to chemical bonds between heavy atoms in the molecule. The node features may include, for example, at least one of an atom type of the heavy atoms, formal charge of the heavy atoms, degree of the heavy atoms, hybridization of the heavy atoms, number of adjacent atoms of the heavy atoms, valence of the heavy atoms, chirality of the heavy atoms, associated ring sizes of the heavy atoms, whether the heavy atoms donate or accept electrons, whether the heavy atoms are aromatic, and whether the heavy atoms contain a ring.

The edge features may include at least one of a bond type of the chemical bonds between the heavy atoms, stereochemistry of the chemical bonds between the heavy atoms, whether the chemical bonds between the heavy atoms contain a ring, and whether the chemical bonds between the heavy atoms contain conjugation.

In one or more examples, the “bond type” of the chemical bonds may refer to a type of force or bond that acts between constituent atoms in an atomic assembly. The bond type may include, but is not necessarily limited to, a covalent bond, ionic bond, hydrogen bond, metallic bond, coordinate covalent bond, Van der Waals force (dispersion force), and hydrophobic bond. The covalent bond may be a bonding state in which two atoms share a pair of electrons in an orbital with each other. The ionic bond may be a bond formed by electrostatic attraction between a positive ion and a negative ion, with electrons gained or lost. The hydrogen bond may be a bond that acts between fluorine (F)/oxygen (O)/nitrogen (N) with high electronegativity and hydrogen (H). The metallic bond may be a bond formed by electrical attraction between electrons and ions that are evenly distributed in a metal. The metallic bond may be a chemical bond that gives metals many metal properties, such as intensity, malleability, ductility, luster, thermal conductivity, and electrical conductivity. The coordinate covalent bond may be a bond in which when two atoms form a covalent bond, the electrons involved in the bond are formally provided by only one atom. The Van der Waals force may refer to a bond that is formed when electrons are locally concentrated within a nonpolar molecule, causing a charge, and an attractive force acts between molecules. The hydrophobic bond may be a force that occurs between nonpolar molecules in water, where water molecules may align around a hydrophobic portion of the molecule due to the hydrophobic bond. The stereochemistry may represent a 3D structure of a molecule or a phenomenon related thereto and may refer to information that considers a spatial arrangement of atom(s) or atomic groups contained in a molecule in three dimensions. The conjugation may refer to alternating single bonds and double bonds (or multiple bonds), as in benzene, for example.

When the products, reactants, and reagents do not have a molecular graph form, the prediction apparatus may generate a 3D molecular structure of at least one element among the products, reactants, and reagents included in the query, for example, in a graph form including nodes and edges.

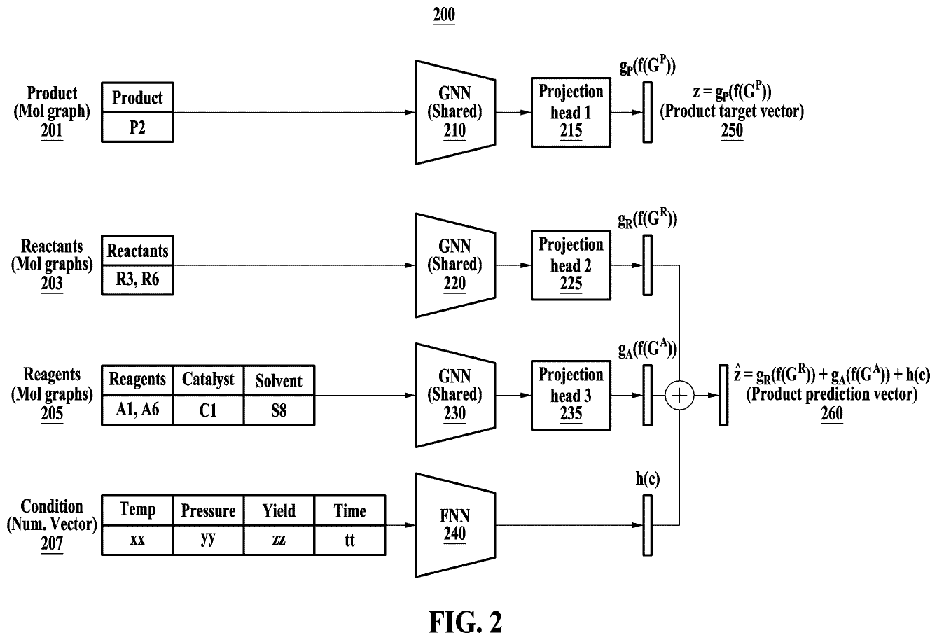

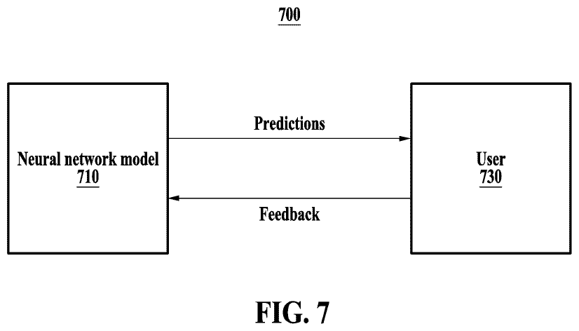

In operation 120, the prediction apparatus may predict (or generate) a query embedding vector corresponding to the query by inputting the query received in operation 110 to a learned (e.g., trained) neural network model (e.g., a neural network model 200 of FIG. 2, a neural network model 320 of FIG. 3A, and/or a neural network model 710 of FIG. 7). The neural network model may include, for example, at least one graph neural network (GNN), and a feed-forward neural network (FNN), but is not necessarily limited thereto. According to embodiments, the at least one GNN may be replaced by a large language model (LLM), or any other suitable neural network model known to one of ordinary skill in the art.

The neural network model may be trained to generate a target vector corresponding to a product for each synthesis recipe and a prediction vector corresponding to at least one of a reactant, a reagent, and a reaction condition. Furthermore, the neural network model may be trained to predict or generate a synthesis recipe corresponding to a query or an embedding vector of the synthesis recipe based on the target vector and the prediction vector. As will be described in more detail below, the embedding vectors of synthesis recipes may be used to learn and visualize embedding vectors that may be used to draw organic synthesis maps, which are two-dimensional (2D) reaction maps for large-scale chemical reaction databases. The structure and operation of the neural network model are described in more detail below with reference to FIG. 2.

In operation 130, the prediction apparatus may extract a candidate embedding vector from among a plurality of embedding vectors based on a similarity between the query embedding vector predicted in operation 120 and a plurality of embedding vectors corresponding to reaction records stored in the database. The prediction apparatus may calculate a similarity score based on a Euclidean distance between the query embedding vector and the plurality of embedding vectors. The similarity score may also be referred to as a “relevance score” in that the similarity score indicates relevance with the query embedding vector.

The prediction apparatus may extract the candidate embedding vector from among the plurality of embedding vectors based on the similarity score. The number of candidate embedding vectors may be, for example, singular or plural. The number of candidate embedding vectors may be K (e.g., K=10), but is not necessarily limited thereto.

The prediction apparatus may select the top K (e.g., K=10) candidate embedding vectors having a high similarity score with the query embedding vector predicted in operation 120 among the reaction records stored in the database. The prediction apparatus may extract candidate reaction records corresponding to K candidate embedding vectors from the database. In one or more examples, the database may be a large-scale chemical reaction database (e.g., the chemical reaction database 350 of FIG. 3A).

In operation 140, the prediction apparatus may output a synthesis recipe corresponding to the query by retrieving a candidate reaction record corresponding to the candidate embedding vector extracted in operation 130 in the database.

The prediction apparatus may visualize synthesis recipes corresponding to a query by displaying candidate reaction records corresponding to candidate embedding vectors on an organic synthesis map. In one or more examples, the candidate embedding vectors displayed on the organic synthesis map may correspond to synthesis recipes that exhibit chemical reactions similar to the query embedding vector.

The prediction apparatus may visualize the synthesis recipes by displaying the synthesis recipes on the organic synthesis map, output each reaction record corresponding to each synthesis recipe as an individual record, or output the reaction records corresponding to each synthesis recipe as a list.

The prediction apparatus may continuously update the neural network model based on user feedback on the output synthesis recipes. A method by which the prediction apparatus updates the neural network model based on user feedback is described in more detail with reference to FIG. 3A below.

The prediction apparatus may automatically perform a design of an experiment, which is conventionally performed manually by a user (e.g., a researcher) to experiment on a particular material that he or she desires to synthesize, by learning experimental recipe data by a pre-trained neural network model and generating and visualizing an organic synthesis map.

The prediction apparatus may be implemented to automatically designate a synthesis path, a range of optimal synthesis recipes, and the like, through a neural network model, and to enable synthesis experiments according to the synthesis recipes predicted by the neural network model through actual automated experimental equipment.

The prediction apparatus may advantageously check for missing information in experimental data by the synthesis recipe output by the neural network model, process a similarity search in a short time, and prevent and/or reduce an increase in search cost due to a search for partial structures of products and/or reactants.

In addition, the prediction apparatus may consider actual values of reaction conditions such as temperature, pressure, and ratio when predicting a synthesis recipe, and may apply the user's knowledge to the neural network model through feedback.

FIG. 2 illustrates a structure and an operation of a neural network model according to one or more embodiments. Referring to FIG. 2, the structure of a neural network model 200 according to one or more embodiments is illustrated.

The neural network model 200 may perform representation learning through contrastive learning, where a model is trained to differentiate between similar and dissimilar data points. In one or more examples, “Representation learning” may correspond to a process in which useful features are automatically extracted from data and the neural network model 200 learns the extracted features on its own. Representation learning may be a process in which a machine (or the neural network model 200) learns how to represent data from data without human intuition or interference, which advantageously helps the neural network model 200 to understand complex patterns in the data and generate better prediction models. Representation learning may be widely used in deep neural networks such as a convolutional neural network (CNN) and may be applied in various fields such as image recognition and natural language processing.

Contrastive learning may be a way of learning by emphasizing the differences between different data, which may correspond to a scheme of learning that similar samples (e.g., positive pairs) come closer to each other and different samples (e.g., negative pairs) move away from each other. Contrastive learning may help the neural network model 200 understand similarities and differences between data and may be useful for representation learning.

The neural network model 200 may include, for example, at least one GNN 210, 220, and 230, projection heads g_{P}, g^{R}, g_{A} 215, 225, and 235 and an FNN h 240. In one or more examples, a projection head may be a small, dedicated neural network layer (e.g., a multi-layer perceptron (MLP)), that takes output features from a main network (e.g., GNN 210, 220, and 230) and projects them into a lower-dimensional space. Projection heads may be used for tasks like contrastive learning, where the goal is to compare similarities between different data points by analyzing their projected representations in this new space. Accordingly, the projection heads help the network focus on the most relevant features for a specific task by transforming the data into a more suitable representation.

When molecules representing structure information of molecules are input to the at least one GNN 210, 220, and 230 through queries, the at least one GNN 210, 220, and 230 may output latent vectors (e.g., a target vector z 250 and a prediction vector \hat{z} 260) that are representation vectors of molecular units corresponding to a molecular graph (Mol graph) G (e.g., G_{P}, G_{R}, and G_{A}) of the input molecules through the projection heads 215, 225, and 235.

The at least one GNN 210, 220, and 230 may include an encoder f that extracts a representation vector of a molecular unit corresponding to each of a product 201, reactants 203, and reagents 205. The encoder f may output a representation vector, which is an embedding vector corresponding to the molecular graph, when molecular graphs G_{P}, G_{R}, and G^{A} corresponding to each of the product 201, reactants 203, and reagents 205 are input into the encoder f.

The at least one GNN 210, 220, and 230 may be composed of a single GNN that shares the encoder f part. Alternatively, the GNN may be composed of a first GNN corresponding to the product 201, a second GNN corresponding to the reactants 203, and a third GNN corresponding to the reagents 205. According to embodiments, each of the at least one GNN 210, 220, and 230 may share parameters of the encoder f for efficient learning.

When there is a missing element (e.g., when an input of the encoder f is missing) among the product 201, reactants 203, and reagents 205, the at least one GNN 210, 220, and 230 may be configured such that the embedding vector output by the projection head corresponding to the missing element may also be set to be a zero vector.

The at least one GNN 210, 220, and 230 may provide, when a missing element exists among the product 201, the reactants 203, and the reagents 205, a distribution value of a neighboring reaction record adjacent to a reaction record corresponding to the missing element in the database as a reaction record value corresponding to the missing element.

The projection heads G_{P}, G_{R}, and G_{A} 215, 225, and 235 connected to the at least one GNN 210, 220, and 230 may be individually configured to correspond to each of the product 201, reactants 203, and reagents 205.

The projection heads G_{P}, G_{R}, and G_{A} 215, 225, and 235 may be composed of, for example, fully-connected layers.

The projection heads G_{P}, G_{R}, and G_{A} 215, 225, and 235 may correspond to the at least one GNN 210, 220, and 230, respectively, and may output a high-dimensional molecular representation vector g (e.g., g_{P}[f[G^{P}]], g_{R}[f[G^{R}]] and g_{A}[f[G^{A}]]) corresponding to the representation vector of a molecular unit output by the at least one GNN 210, 220, and 230.

The projection heads g_{P}, g_{R}, g_{A} 215, 225, and 235 may include at least one of, for example, a first projection head g_{P} 215 that converts a first representation vector corresponding to the product 201 to a first latent vector g_{P}[f[G^{P}]], a second projection head g_{R} 225 that converts a second representation vector corresponding to the reactants 203 to a second latent vector g_{R}[f[G^{R}]], and a third projection head g_{A} 235 that converts a third representation vector corresponding to the reagents 205 to a third latent vector g_{R}[f[G^{R}]], but are not necessarily limited thereto. The projection heads g_{P}, g_{R}, g_{A} 215, 225, and 235 may output a projected vector in which the input embedding vector is projected into a low dimension.

The first projection head g_{P} 215 may convert the representation vector of a molecular unit corresponding to the product (e.g., P2) 201 to a low-dimensional latent vector. The second projection head g_{R} 225 may convert the representation vector of a molecular unit corresponding to the reactants (e.g., R3 and R6) 203 to a low-dimensional latent vector. The third projection head 9A 235 may convert the representation vector of a molecular unit corresponding to the reagents (e.g., reagents: A1 and A6, catalyst: C1, solvent: S8) 205 to a low-dimensional latent vector.

The projection heads g_{P}, g_{R}, g_{A} 215, 225, and 235 may output a zero vector in response to a missing element when a missing element exists among the product 201, reactants 203, and reagents 205 input to the encoder f of the at least one GNN 210, 220, and 230. This may be for zero imputation for a missing element when there are missing elements. When a value corresponding to a missing element is replaced with “0”, the distribution of data may be distorted, however, a variable with a value of “0” may not affect the embedding vector. “Zero imputation” may be a method for handling a missing value, and may correspond to a technique for replacing a missing value with “0”. The completeness of a data set may be maintained by filling in missing values with “0” according to zero imputation. When zero imputation is used, the implementation may be simple, reducing computational cost, and the size of the data set may be maintained without removing the missing values.

The FNN h 240 may have a structure in which an input value is transmitted in one direction to the output, and may correspond to an artificial neural network with one or more hidden layers. In the FNN h 240, data moves unidirectionally from the input layer of a neural network to the output layer, so no recurrence or feedback may occur. The FNN h 240 may also be referred to as a “multi-layer perceptron (MLP).”

The FNN h 240 may output a reaction vector h[c] corresponding to a condition vector c in accordance with the condition vector c corresponding to a reaction condition 207 being input. The reaction condition 207 may include, but is not necessarily limited to, temperature, pressure, yield (e.g., reaction yield), reaction time, and reaction type. Each of the layers (e.g., hidden layer(s)) of the FNN h 240 may be set with a bias value or weight. The neural network model 200 may set the bias values for all layers of the FNN 240 processing the reaction vector h[c] corresponding to the reaction condition 207 to be “0”. The bias values may be dynamically determined during the training and updating of the neural network model 200.

For example, when a condition vector c corresponding to the reaction condition 207 equals [0, 0, 0, 0], the reaction vector h[c], which is the resulting embedding vector, may also be a zero vector.

The neural network model 200 may predict the first latent vector g_{P}[f[G^{P}]] to be a target vector z=g_{P}[f[G^{P}]] by applying the first representation vector corresponding to the product 201 to the first projection head g_{P} 215. The first latent vector g_{P}[f[G^{P}]] may correspond to a latent vector corresponding to the product 201.

The neural network model 200 may predict the second latent vector g_{R}[f[G^{R}]] by applying the second representation vector corresponding to the reactants 203 to the second projection head g_{R} 225. The second latent vector g_{R}[f[G^{R}]] may correspond to a latent vector corresponding to the reactants 203.

The neural network model 200 may predict the third latent vector g_{A}[f[G^{A}]] by applying the third representation vector corresponding to the reagents 205 to the third projection head g_{A} 235. The third latent vector g_{A}[f[G^{A}]] may correspond to a latent vector corresponding to the reagents 205.

The neural network model 200 may apply the reaction condition 207 to the FNN 240 to extract the reaction vector h[c] corresponding to the reaction condition 207.

The neural network model 200 may further include a one-hot-encoding layer corresponding to each of the first projection head 215, the second projection head 225, the third projection head 235, and the FNN 240.

The neural network model 200 may generate a prediction vector \hat{z} 260 based on at least one of the second latent vector g_{R}[f[G^{R}]], the third latent vector g_{A}[f[G^{A}]] and the reaction vector h[c].

The neural network model 200 may add up the second latent vector g_{R}[f[G^{R}]], the third latent vector g_{A}[f[G^{A}]] and the reaction vector h[c] such as \hat{z}=g_{R}[f[G^{R}]]+g_{A}[f[G^{A}]]+h[c], to generate the prediction vector \hat{z} 260.

The neural network model 200 may be trained to predict a synthesis recipe corresponding to a query or an embedding vector of a synthesis recipe based on the target vector z 250 and the prediction vector \hat{z} 260. In one or more examples, the embedding vector of the synthesis recipe may be used by the prediction apparatus to visualize the organic synthesis map. The neural network model 200 may generate an organic synthesis map, which is a 2D reaction map for a large-scale chemical reaction database.

The neural network model 200 may be trained to predict a target recipe corresponding to the product 201, or may be trained to predict a target recipe corresponding to the reactants 203 and the reaction condition 207 (or the reactants 203, the reagents 205 and the reaction condition 207). The neural network model 200 may be trained by contrastive learning based on the target vector z 250 and the prediction vector \hat{z} 260.

A minibatch

$$B = {\{ ( {G}^{P} , {G}^{R} , {G}^{A} , c ) \}}_{i = 1}^{M}$$

consisting of M reaction records may be given during an iteration of the learning process. When a training data set such as a minibatch is given, the neural network model 200 may perform training on the at least one GNN 210, 220, and 230 to minimize an objective function J based on a loss function l.

The neural network model 200 may generate the target vector zᵢ and the prediction vector \hat{z}ᵢ for each of the M reaction records. Accordingly, a total of 2M vectors {z₁, . . . , z_{M},\hat{z}₁, . . . , \hat{z}_{M}} may be used to train the neural network model 200.

The neural network model 200 may use the 2M vectors {z₁, . . . , z_{M},\hat{z}₁, . . . , \hat{z}_{M}} to configure the positive pairs and negative pairs for the contrastive learning described above. The “positive pair” may be a pair (zᵢ, \hat{z}ᵢ) of a target vector and a prediction vector of the same reaction record, and i=1, . . . , M. In one or more examples, the same reaction record may be a reaction record(s) corresponding to the same synthesis recipe among reaction records stored in the database. The “negative pair” may correspond to a pair corresponding to all reaction records except the positive pair among the reaction records stored in the database.

The neural network model 200 may learn a representation that causes the positive pairs to come closer to each other and the negative pairs to move away from each other through contrastive learning. In one or more examples, the positive pairs coming closer to each other may indicate that a distance (the Euclidean distance) between the target vector zᵢ, and the prediction vector \hat{z}ᵢ that make up the positive pairs in a vector space becomes closer (e.g., value of Euclidean distance is becoming smaller). In addition, the “the negative pairs move away from each other” may indicate that a distance (the Euclidean distance) between the target vector zᵢ, and the prediction vector \hat{z}ᵢ that make up the negative pairs in the vector space is getting farther (e.g., value of Euclidean distance is becoming larger).

For example, when expressed as & an example, a contrastive loss function for contrastive learning of the neural network model 200 may be expressed by Equation 1 below.

$$l ( i , j ) = - log \frac{exp ( - d ( {z}_{i} , {z}_{j} ) / 𝒯 )}{{\sum }_{k = 1}^{2 M} 1 ( i \neq k ) exp ( - d ( {z}_{i} , {z}_{k} ) / 𝒯 )} \qquad [ Equation 1 ]$$

In one or more examples, Z (e.g., zᵢ, zⱼ, zₖ)) denotes a representation vector value for each reagent as shown in FIG. 2. Also, τ denotes a temperature hyperparameter, and d denotes a distance function (e.g., a squared Euclidean distance). The neural network model 200 may calculate a distance between two embedding vector values based on a negative log value of zᵢ, zⱼ. In one or more examples, a learning object or objective function J of the neural network model 200 may be expressed by Equation 2 below. Equation 2 may set the objective function such that the neural network model 200 learns in the direction of a small difference when the difference between zᵢ, zⱼ that minimizes the loss value derived in Equation 1 is a positive pair, and in the direction of a large difference when the difference is a negative pair.

$$𝒥 = \frac{1}{M} {\sum }_{i = 1}^{M} [ l ( i , M + i ) + l ( M + i , i ) ] \qquad [ Equation 2 ]$$

In one or more examples, M denotes the number of reaction records, and i denotes an embedding vector value of an i-th product structure. M+i denotes an embedding vector r value of a reagent corresponding to the i-th product.

In one or more embodiments, by applying M+i to the loss l of Equation 1 such as l(i, M+i) and l(i, M+i,i) in a cross-like manner, the neural network model 200 may be trained such that positive pairs become closer together and negative pairs become further apart. In one or more examples, since two values are added, a value divided by ½ may be an objective function J, and by training the neural network model 200 to minimize the objective function J, the embedding vectors of the positive pair may be generated to be closer together, and the embedding vectors of the negative pair may be generated to be farther apart from each other.

In one or more embodiments, the synthesis experiment data may be visualized after actual synthesis through visualization of an organic synthesis map, or the actual synthesis experiment results may be reflected in the neural network model 200 to provide search results tailored to the convenience of a user.

FIG. 3A illustrates a process of updating a neural network model by reflecting user feedback according to one or more embodiments.

Referring to FIG. 3A, an example in which a neural network model 320 (e.g., the neural network model 200 of FIG. 2 and/or the neural network model 710 of FIG. 7) according to one or more embodiments receives a query 315 of a user 310, shows search results 340 corresponding to the query 315, and stores an evaluation result including the search result 340 in a query database (query DB) 330 according to the user 310 evaluating the search result 340 through feedback 345 and reflects the evaluation result in training data 360 and updating 380 of the neural network model 320.

For example, when the query 315 includes both a product and reactants, the search result 340 of the neural network model 320 may be a synthesis recipe including a synthesis path, synthesis conditions, and the like, for generating a product from the reactants. When the query 315 includes reactants, reaction conditions, and reagents, the search result 340 of the neural network model 320 may be a product that may be synthesized by the reactants, reaction conditions, and reagents. When the query 315 includes a product, the search results 340 of the neural network model 320 may be reactants, reaction conditions, and reagents that may be used to produce the product.

For example, a training apparatus may periodically update (380) the neural network model 320 by considering the search results 340, the query DB 330, and/or the training data 360 updated by the feedback 345 of the user, in order to update (380) the neural network model 320 by the feedback 345 of the user 310.

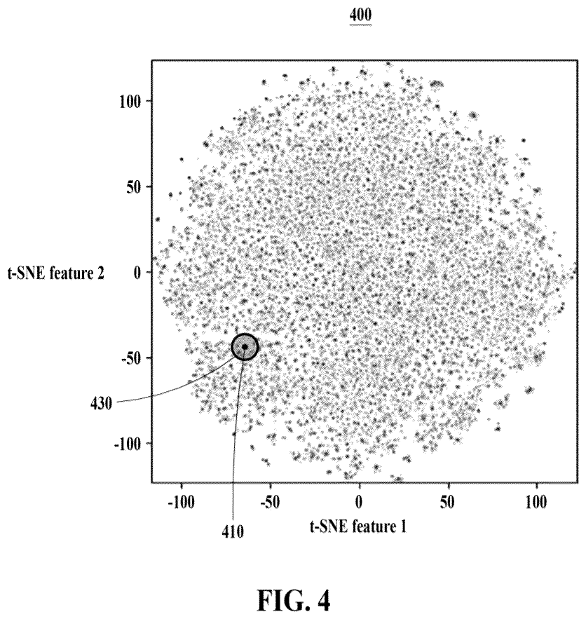

For example, after the neural network model 320 is trained, and an embedding vector for each synthesis recipe as the search result 340 is generated, the user may input the query 315 corresponding to a synthetic material to be searched for (e.g., a target material). The neural network model 320 may visualize a list of synthesis recipes most similar to the query 315 in the form of a table such as the search results 340 or in the form of a 2D map as illustrated in FIG. 4.

The user 310 may provide the feedback 345 on an evaluation result or preference for a synthesis recipe included in the visualized table or 2D map. The training apparatus may update the query DB 330 by reflecting the feedback 345 evaluation result or preference, and sample 353 reaction records stored in a chemical reaction database 350 according to the information in the updated query DB 330. The training apparatus may update the training data 360 by reflecting the sampled reaction records 353 and the information in the query DB 330. The neural network model 320 may be periodically updated 380 by the updated training data 360 to reflect the evaluation result or preference of the user when the next synthesis recipe is to be retrieved or predicted.

The training apparatus may, for example, use a modified objective function I to periodically perform updates 380 on the neural network model 320. The objective function I may be an objective function of contrastive learning using reaction records randomly sampled 353 from the chemical reaction database 350.

The training apparatus may use the most recent L reaction records stored in the query DB 330 as samples 353. A candidate embedding vector extracted from the query DB 330 may be expressed as x*, and K reaction embedding vectors retrieved from the chemical reaction database 350 by the neural network model 320 may be expressed as x₍ᵢ₎*.

The training apparatus may fine-tune the neural network model 320 so that a relative distance (e.g., the Euclidean distance) between the candidate embedding vector x* and the reaction embedding vector x₍ᵢ₎* in the vector space satisfies Equation 3 below.

$$\begin{matrix}if {r}_{( i )} > {r}_{( j )} , then d ( {x}^{*} , {x}_{( i )}^{*} ) < d ( {x}^{*} , {x}_{( j )}^{*} ) \\ if {r}_{( i )} < {r}_{( j )} , then d ( {x}^{*} , {x}_{( i )}^{*} ) > d ( {x}^{*} , {x}_{( j )}^{*} )\end{matrix} \qquad [ Equation 3 ]$$

In one or more examples, x* denotes a query corresponding to a product or recipe that the user is looking for, and r(i) denotes a rating.

Equation 3 may represent “human-in-the-loop”, that is, a process of reflecting the user's rating of a result to the neural network model 320. For examples, when a query x* is input, the top-K results may be x*1, . . . , x*k, and the preferences may be given as +1 for a positive pair, −1 for a negative pair, and 0 for neutral or no response, which may be expressed as x*(i). The parameter r*(i) may have one of three values: −1, 0, +1. The training apparatus may update the neural network model 320 based on the evaluation of the top-k results for the query (x*) value.

In addition, a modified objective function \tilde{J} based on the Ranking Loss may be expressed by Equation 4 below.

$$\tilde{𝒥} = 𝒥 + λ \cdot \frac{1}{\vert Q \vert} {\sum }_{{x}^{*} ∈ Q} [ \frac{2}{K ( K - 1 )} {\sum }_{i = 1}^{K - 1} {\sum }_{j = i + 1}^{K} max ( 0 , ( {r}_{( i )}^{*} - {r}_{( j )}^{*} ) \cdot ( d ( {x}^{*} , {x}_{( i )}^{*} ) - d ( {x}^{*} , {x}_{( j )}^{*} ) ) ) ] \qquad [ Equation 4 ]$$

In one or more examples, Q denotes a query set, and K denotes the number of pieces of data. In addition, λ denotes an eigenvalue obtained by a principal component analysis (PCA). Equation 4 may indicate that the neural network model 320 may be updated by reflecting the objective function for processing the above-described positive pair, negative pair, and neutral.

FIG. 3B illustrates a process of generating an organic synthesis map based on search results corresponding to a user query according to one or more embodiments. Referring to FIG. 3B, a result of a list of candidate synthesis recipes including a synthesis recipe showing the highest similarity to the query 315 visualized in the form of a 2D map 370 by candidate embedding vectors corresponding to the synthesis recipe is illustrated.

As illustrated in FIG. 3B, when the query 315 is input, the neural network model 320 may retrieve a reaction record corresponding to a target material included in the query 315 in the chemical reaction database 350. In one or more examples, the target material may be a product or may be a reactant and a reaction condition (or a reactant, a reagent, and a reaction condition).

For example, when the target material included in the query 315 is a product, the neural network model 320 may search for a reaction record 355 corresponding to the highest rank (e.g., Rank=1) among the search results 340 retrieved from the chemical reaction database 350 based on a similarity score with the target material (“product”) included in the query 315. In one or more examples, the reaction record 355 may correspond to one of the lists of synthesis recipes most similar to the query 315. The reaction record 355 may include, but is not necessarily limited to, information on identification information (e.g., ID=R12345) of a corresponding synthesis recipe, a link (a uniform resource locator (URL)) to an experimental paper similar to the synthesis recipe, a yield (e.g., 36.7%), reactants (e.g., R3 and R6), a product (e.g., P2), reaction temperature (e.g., xx degrees), reaction pressure (e.g., yy atm), catalyst (e.g., C1), solvent (e.g., S8), and/or reagents (e.g., A1 and A6) of the synthesis recipe. The neural network model 320 may display the synthesis recipe corresponding to the retrieved target material and candidate recipes with a high similarity to the synthesis recipe together with the target material on the 2D map 370.

When the target material included in the query 315 is a reactant and a reaction condition, the neural network model 320 may search for the reaction record 355 corresponding to the highest rank among the search results 340 retrieved from the chemical reaction database 350 based on a similarity score between the reactant and the reaction condition included in the query 315.

According to one or more embodiments, the neural network model 320 may be installed in a web-based virtual synthesis tool. When a user inputs a desired target material through the query 315, the neural network model 320 may search for experimental papers and results similar to the target material and provide them as the search results 340. The experimental papers may be provided, for example, in the form of a paper URL. In addition, the neural network model 320 may enable experiments using actual automated experimental equipment by allowing the user to automatically specify a synthesis path corresponding to a target material, and/or an optimal recipe range, through the query 315.

The neural network model 320 may allow the user to rate each individual [symbol] reaction record corresponding to the search results 340 as “like” (e.g., [symbol] ) or “dislike” (e.g., [symbol] ), or no response (e.g., [symbol] ), and by receiving the user's rating as feedback 345 and reflecting the feedback 345 in the learning of the neural network model 320, continuous performance improvement may be possible.

FIG. 3C illustrates a method of extracting reaction records by similarity scores when a user query is given according to one or more embodiments. Referring to FIG. 3C, a diagram showing a prediction apparatus according to one or more embodiments extracting N candidate reaction records corresponding to the query 315 from the chemical reaction database 350 based on a similarity score when the query 315 is given is illustrated. In one or more examples, the N candidate reaction records may correspond to candidate embedding vectors corresponding to the query 315.

For example, when the query 315 includes both reactants and a product, the candidate embedding vector corresponding to the query 315 may be expressed as x*=[z*][\hat{z}*]. When the query 315 includes a product, the candidate embedding vector corresponding to the query 315 may be expressed as x*=z*. When there are two or more reaction records having the same product in the chemical reaction database 350, the prediction apparatus may select a reaction record in order of higher yield among the two or more reaction records.

When the query 315 includes reactants (or reactants and reaction conditions), the candidate embedding vectors may be expressed as x*=\hat{z}*.

The prediction apparatus may extract N reaction records having a high similarity score with the query embedding vector among the embedding vectors stored in the chemical reaction database 350. In one or more examples, the similarity score may be obtained by, for example, Score=exp(−0.0001·^{d(x*,x}^{ᵢ})).

FIG. 3D illustrates an example of candidate embedding vectors stored in a query database according to one or more embodiments. Referring to FIG. 3D, an example of the query DB 330 in which K search results with high similarity scores are stored for each query according to one or more embodiments is illustrated.

The query DB 330 may include K (e.g., K=“10” or K=“15”) search results (e.g., reaction records) with high similarity for each query ID. Each search result may include K reaction records for each query ID. The reaction records may include, for example, an ID, product ID, condition ID, and user evaluation items.

The user evaluation may have values such as positive (“+1”), negative (“−1”), and no response (or neutral) (“0”), or values based on more detailed classifications (e.g., strong positive (“+1”), weak positive (“+0.5”), weak negative (“−0.5”), strong negative (“−1”), and no response (“0”)).

FIG. 4 illustrates a visualized result of searching for synthesis recipes corresponding to a query according to one or more embodiments. Referring to FIG. 4, a 2D map 400 visualizing synthesis recipes according to one or more embodiments is illustrated.

A prediction apparatus may display, in response to a query of a user, a synthesis recipe 410 predicted by a pre-trained neural network model (e.g., the neural network model 200 of FIG. 2, the neural network model 320 of FIG. 3A, and/or the neural network model 710 of FIG. 7) and embedding vectors corresponding to similar synthesis recipes 430 adjacent to (similar to) the synthesis recipe 410 on the 2D map 400. The 2D map 400 may correspond to a map that embeds the entire database (e.g., the chemical reaction database 350 of FIG. 3) in a 2D space.

The prediction apparatus may show the top-K reaction records with high similarity to the query and locations of the top-K reaction records in the entire 2D map 400. The synthesis recipes 430 adjacent to (similar to) the synthesis recipe 410 may correspond to embedding vectors of the top-K reaction records with high similarity to the query.

The user may view and provide feedback on the similar synthesis recipes 430 displayed on the 2D map 400 so that the preferences or evaluation results of the user are reflected in the neural network model.

The prediction apparatus may reduce the space of the 2D map 400 using, for example, a T-distributed Stochastic Neighbor Embedding (T-SNE) algorithm, but is not necessarily limited thereto. In the 2D map 400, a distance indicator may correspond to the Euclidean distance, the same as the setting during learning.

The T-SNE algorithm may be a nonlinear dimensionality reduction scheme for reducing high-dimensional complex data to two or three dimensions. The T-SNE algorithm may be mainly used for low-dimensional spatial visualization, and since data is organized by similar structures when the dimension is reduced, it may help understand the data structure. The T-SNE algorithm may calculate a similarity between points in a high-dimensional space and the corresponding similarity between points in a low-dimensional space. The similarity of the points may be calculated as a conditional probability that point A selects point B as a neighbor, for example, when neighbors are selected in proportion to their probability density from a normal distribution centered at point A. In one or more examples, a difference between the conditional probabilities (or similar points) in the high-dimensional space and the low-dimensional space may be minimized to perfectly represent the data elements in the low-dimensional space. To minimize a sum of the differences between the conditional probabilities, the T-SNE algorithm may use a gradient descent scheme to minimize a sum of Kullback-Leibler (KL)-divergences across all data points.

The T-SNE algorithm may be one of manifold learning algorithms that may visualize complex data, such as high-dimensional data, by reducing the data to two or three dimensions. In one or more examples, similar data structures in a high-dimensional space may correspond closely in a low-dimensional space, and dissimilar, or in other words different, data structures may correspond far apart in a low-dimensional space.

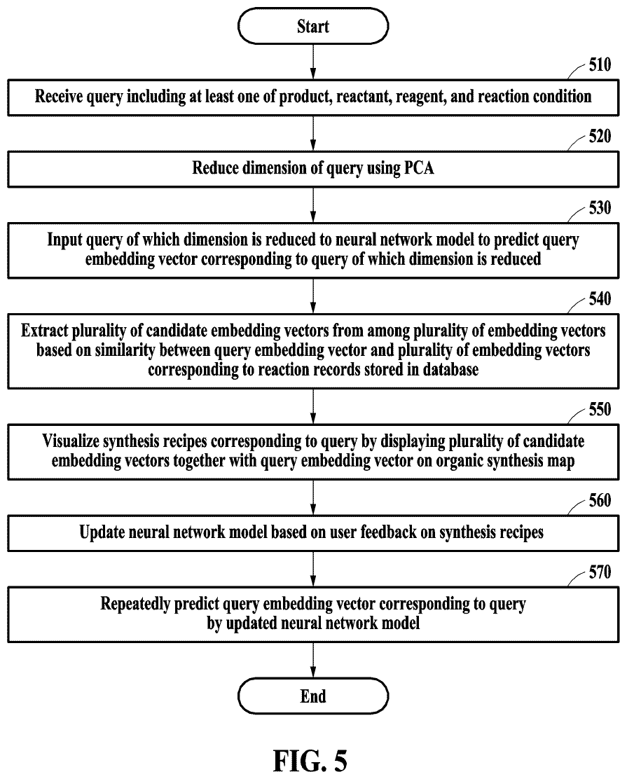

FIG. 5 is a flowchart of a method of predicting a synthesis recipe according to one or more embodiments. Referring to FIG. 5, a prediction apparatus according to one or more embodiments may repeatedly predict a query embedding vector corresponding to a query through operations 510 to 570.

In operation 510, the prediction apparatus may receive a query including at least one of a product, a reactant, a reagent, and a reaction condition. When the product, reactant, and reagent do not have a molecular graph form, the prediction apparatus may perform preprocessing to represent a 3D molecular structure of at least one element among the product, reactant, and reagent included in the query in a graph form.

In operation 520, the prediction apparatus may reduce a dimension of the query received in operation 510 using principal component analysis (PCA). The prediction apparatus may perform dimensionality reduction through PCA before distance calculation for approximate distance calculation. In one or more examples, “PCA” may be an approach to find the principal components of data included in the query. PCA may not be an approach to analyze the components of each data, but rather an approach to analyze the principal components of a distribution when multiple data come together to form a distribution. A “principal component” may refer to a direction vector corresponding to a direction in which the variance of data in a distribution is the greatest. In one or more examples, PCA may implement a linear dimensionality reduction technique, where data is linearly transformed onto a new coordinate system such that the directions capturing a largest variation in data is identified. The prediction apparatus may, for example, perform PCA on a 2D data set included in a query and output two mutually perpendicular principal component vectors. Furthermore, the prediction apparatus may perform PCA on 3D points included in the query and output three mutually perpendicular principal component vectors. The prediction apparatus may shorten a prediction time for the query embedding vector by reducing the dimension of the query before distance calculation through PCA.

A process by which the prediction apparatus reduces the dimension of the query through PCA may be as follows.

The prediction apparatus may configure a matrix of a target vector Z₁ corresponding to the query, and a matrix of a prediction vector Z₂ to be Z₁=[z₁;z₂; . . . ; z_{N}]; Z₂=[\hat{z}₁;\hat{z}₂; . . . ; \hat{z}_{N}];

The prediction apparatus may perform an r-dimensional low-rank Singular Value Decomposition (SVD) on each d-dimensional matrix. SVD may reduce data so that only key features, or in other words, principal components necessary to analyze the data (e.g., the query) remain.

When the principal components of the target vector Z₁ and the prediction vector Z₂ are V1 and V2, respectively, the prediction apparatus may reduce the dimension of the target vector Z₁ and the prediction vector Z₂ as follows: zᵢ′=zᵢV₁;\hat{z}ᵢ′=zᵢV₂. The prediction apparatus may reduce a search time for an individual query by a factor of r/d by reducing the dimension of the query from d to r (r<<d). Also, zᵢ∈ [symbol] ^{d}, and zᵢ′∈ [symbol] ^{r}, r<<d may be established.

In operation 530, the prediction apparatus may input the query of which the dimension is reduced in operation 520 to a neural network model (e.g., the neural network model 200 of FIG. 2, the neural network model 320 of FIG. 3A, and/or the neural network model 710 of FIG. 7) to predict a query embedding vector corresponding to the query of which the dimension is reduced. The neural network model may be trained to generate a target vector corresponding to a product for each synthesis recipe and a prediction vector corresponding to at least one of a reactant, a reagent, and a reaction condition, and to predict an embedding vector of a synthesis recipe corresponding to a query based on the target vector and the prediction vector. The embedding vector of the synthesis recipe may be used to learn an embedding vector configured to draw an organic synthesis map and to visualize the organic synthesis map.

In operation 540, the prediction apparatus may extract a plurality of candidate embedding vectors from among a plurality of embedding vectors based on a similarity between the query embedding vector predicted in operation 530 and a plurality of embedding vectors corresponding to reaction records stored in a database. The prediction apparatus may calculate a similarity score based on the Euclidean distance between the query embedding vector and the plurality of embedding vectors. The prediction apparatus may extract the plurality of candidate embedding vectors from among the plurality of embedding vectors based on the similarity score. The prediction apparatus may extract candidate reaction records corresponding to the top K (e.g., K=10) candidate embedding vectors having a high similarity score among the reaction records stored in the database. In one or more examples, the database may be a large-scale chemical reaction database.

In operation 550, the prediction apparatus may visualize the synthesis recipes corresponding to the query by displaying the plurality of candidate embedding vectors extracted in operation 540 together with the query embedding vector predicted in operation 530 on the organic synthesis map.

In operation 560, the prediction apparatus may update the neural network model based on user feedback on the synthesis recipes visualized in operation 550. The user feedback may correspond to the user's evaluation of the synthetic recipes. The user feedback may include, but is not necessarily limited to, at least one of very like, like, slightly like, slightly dislike, dislike, very dislike, and no response (or don't know). In one or more examples, an update to the neural network model may correspond to a fine-tuning process for the neural network model.

In operation 560, the prediction apparatus may store an evaluation score corresponding to the user feedback in a query database. The prediction apparatus may assign an evaluation score (e.g., very like (1), like (0.5), slightly like (0.2), slightly dislike (−0.2), dislike (−0.5), very dislike (−1), no response or don't know (0)) corresponding to the user feedback (e.g., very like, like, slightly like, slightly dislike, dislike, very dislike, no response or don't know) for a synthesis recipe, and store the assigned evaluation score in a query database corresponding to the synthesis recipe. The prediction apparatus may update the reaction records stored in the database by reflecting the evaluation score on the similarity of the embedding vector corresponding to the corresponding synthesis recipe in the query database. The prediction apparatus may update the neural network model based on the updated reaction records.

In operation 570, the prediction apparatus may repeatedly predict or generate a query embedding vector corresponding to the query by the neural network model updated in operation 560.

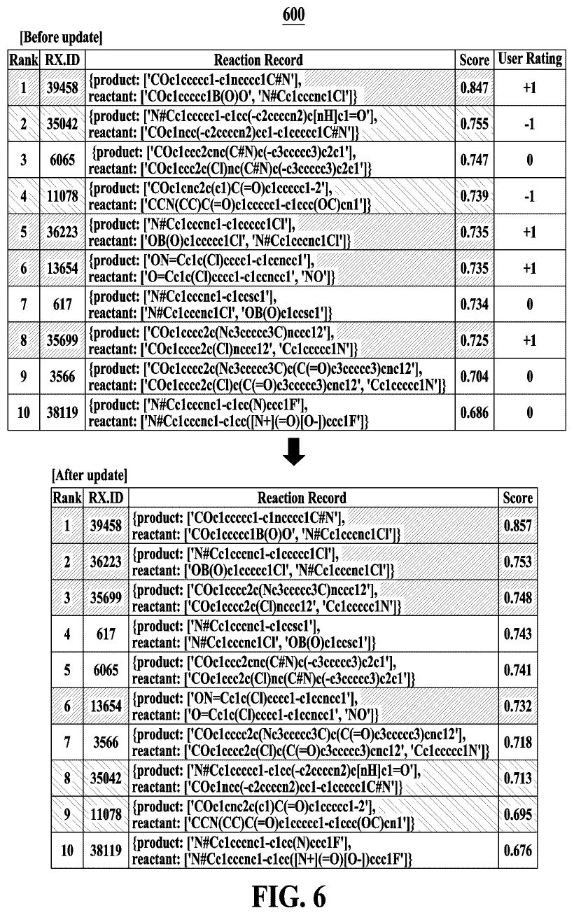

FIG. 6 illustrates search results after a neural network model is updated based on user feedback according to one or more embodiments. Referring to FIG. 6, a table 600 showing search results before (upper table) and after (lower table) a neural network model (e.g., the neural network model 200 of FIG. 2, the neural network model 320 of FIG. 3A, and/or the neural network model 710 of FIG. 7) is updated based on user feedback when a query is {‘product’: ‘Ccclccccc1-clccncc1C #N’, ‘reactant’: ‘COclccccclB(O)O’, ‘N #Cclcccnc1Cl’}, is illustrated.

In the table 600, a similarity score of a reaction record corresponding to a user's rating of “good” or “very good” by feedback may be reflected with an evaluation score of “+1” corresponding to the user's rating, which may increase the search ranking of the corresponding reaction record. The similarity score of a reaction record corresponding to a user's rating of “bad” or “very bad” may be reflected with an evaluation score of “−1” corresponding to the user's rating, which may lower the search ranking of the corresponding reaction record.

Based on these features, feedback on the evaluation results of the user may advantageously influence the performance results of the neural network model, so that the preference of the user is reflected in the search results (or prediction results) of the neural network model.

FIG. 7 illustrates a process in which user feedback is reflected in the prediction results of a neural network model according to one or more embodiments. Referring to FIG. 7, an example 700 of an environment in which a neural network model 710 is continuously updated through evaluation feedback of a user 730 on prediction results of the neural network model 710 (e.g., the neural network model 200 of FIG. 2, and/or the neural network model 320 of FIG. 3A) is illustrated.

The neural network model 710 may be continuously updated by online learning and/or incremental learning. Incremental learning may correspond to machine learning in which additional input data is continuously input while there is a pre-trained neural network model 710. Incremental learning may correspond to a machine learning scheme in which knowledge of the pre-trained neural network model 710 is expanded by additional input data, or in other words, the neural network model 710 is additionally trained by additional input data.

In one or more examples, the additional input data may include expertise from domain experts as well as feedback from the user 730 on a synthesis recipe predicted by the neural network model 710. The neural network model 710 may also provide customized search results to users by separately reflecting feedback (evaluation results) from the individual user 730.

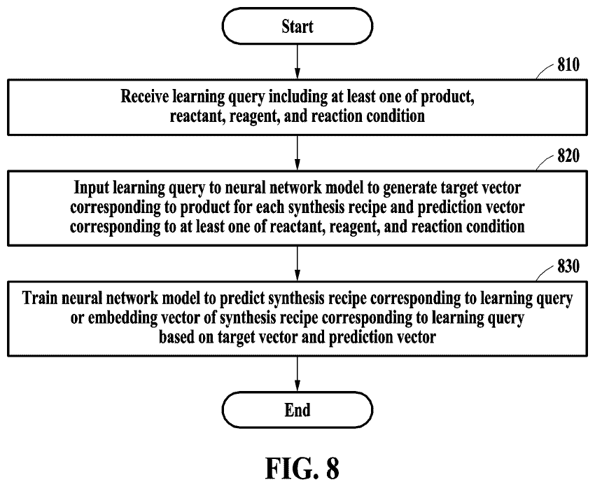

FIG. 8 is a flowchart of a learning method of a neural network model according to one or more embodiments. Referring to FIG. 8, a training apparatus according to one or more embodiments may train a neural network through operations 810 to 830.

In operation 810, the training apparatus may receive a learning query including at least one of a product, a reactant, a reagent, and a reaction condition.

In operation 820, the training apparatus may input the learning query received in operation 810 to the neural network model (e.g., the neural network model 200 of FIG. 2, the neural network model 320 of FIG. 3A, and/or the neural network model 710 of FIG. 7), and generate i) a target vector corresponding to a product for each synthesis recipe and ii) a prediction vector corresponding to at least one of a reactant, a reagent, and a reaction condition. The neural network model may include, for example, at least one GNN, projection heads and an FNN.

The at least one GNN may include an encoder that extracts a representation vector of a molecular unit corresponding to each of a product, reactant, and reagent. The at least one may provide, when a missing element exists among the product, reactant, and reagent, a distribution value of a neighboring reaction record adjacent to a reaction record corresponding to the missing element in a database as a value corresponding to the missing element.

The second projection heads may convert the representation vector of a molecular unit corresponding to each of the product, reactant, and reagent to a low-dimensional latent vector. The projection heads may include at least one of, for example, a first projection head that converts a first representation vector corresponding to the product to a first latent vector, a second projection head that converts a second representation vector corresponding to the reactant to a second latent vector, and a third projection head that converts a third representation vector corresponding to the reagent to a third latent vector, but are not necessarily limited thereto. The projection heads may output a zero vector in response to a missing element when a missing element exists among the product, reactant, and reagent input to an encoder.

An FNN may output a reaction vector corresponding to a reaction condition.

Bias values may be set in the layers of the FNN.

In operation 820, the training apparatus may predict the first latent vector corresponding to the product as a target vector by applying the first representation vector corresponding to the product to the first projection head corresponding to the product. The training apparatus may predict the second latent vector by applying the second representation vector corresponding to the reactant to the second projection head corresponding to the reactant. The training apparatus may predict the third latent vector corresponding to the reagent by applying the third representation vector corresponding to the reagent to the third projection head corresponding to the reagent. The training apparatus may apply a reaction condition to the FNN to extract a reaction vector corresponding to the reaction condition. The training apparatus may generate a prediction vector based on at least one of the second latent vector, the third latent vector, and the reaction vector.

In operation 830, the training apparatus may train the neural network model to predict a synthesis recipe (or an embedding vector of the synthesis recipe) corresponding to the learning query based on the target vector and prediction vector generated in operation 820. In one or more examples, the embedding vector of the synthesis recipe may be used by the prediction apparatus to generate and visualize the organic synthesis map. The training apparatus may train the neural network model to predict a target recipe corresponding to a product.

The training apparatus may train the neural network model by contrastive learning based on the target vector and the prediction vector. The process by which the training apparatus trains the neural network model through contrastive learning may be as follows. The training apparatus may configure a positive pair by a target vector and a prediction vector corresponding to the same synthesis recipe. The training apparatus may configure a negative pair by a target vector and a prediction vector corresponding to a different synthesis recipe. The training apparatus may learn a representation in the neural network model that makes the Euclidean distance of the positive pair closer to each other, and the Euclidean distance of the negative pair farther from each other.

The training apparatus may pre-train a graph neural network using a large-scale chemical reaction database, and perform a target task such as compound prediction using the pre-trained graph neural network, thereby overcoming the performance degradation of material-based prediction models with insufficient amount or variety of training data, while providing a high-performance prediction model even with a small amount of data.

In one or more examples, the training apparatus may receive user feedback on the synthesis recipe corresponding to the learning query. The training apparatus may update the database with additional learning data generated by the user's feedback, and update the neural network model with the updated database. The training apparatus may train the neural network model such that an embedding vector of a synthesis recipe corresponding to a user's “like” feedback is composed of positive pairs, for example. The training apparatus may train the neural network model such that an embedding vector of a synthesis recipe corresponding to a user's “dislike” feedback is composed of negative pairs.

Additionally, when the degree (e.g., very like (strong), slightly like (weak), dislike (strong), slightly dislike (weak), etc.), of a “like” and “dislike” of a user is different, the training apparatus may process strong likes and weak likes using a ranking scheme of the form (+2, +1, 0, −1, −2) in lieu of the three ranking schemes of (+1, 0, −1) in the aforementioned Equation 3.

According to embodiments, the training apparatus may use PCA to reduce the dimensionality of vectors while retaining original information as much as possible to remove redundant information.

FIG. 9 is a block diagram of an apparatus for predicting a synthesis recipe according to one or more embodiments. Referring to FIG. 9, a prediction apparatus 900 according to one or more embodiments may include a communication interface 910, a memory 930, a processor 950, and a display 960. The communication interface 910, the memory 930, and the processor 950 may be connected to each other via a communication bus 905. Although FIG. 9 illustrates that the display is part of the prediction apparatus 900, as understood by one of ordinary skill in the art, the embodiments are not limited to this configuration. For example, the display 960 may be an external display (e.g., computer monitor) that is connected to the prediction apparatus 900.

The communication interface 910 may receive a query including at least one of a product, a reactant, a reagent, and a reaction condition.

The memory 930 may store a neural network model (e.g., the neural network model 200 of FIG. 2, the neural network model 320 of FIG. 3A, and/or the neural network model 710 of FIG. 7). The neural network model 710 may be pre-trained and may include the at least one GNN and FNN described above.

The processor 950 may predict a query embedding vector corresponding to a query by inputting the query received through the communication interface 910 to the neural network model stored in the memory 930. The processor 950 may extract a plurality of candidate embedding vectors from among a plurality of embedding vectors based on a similarity between the query embedding vector and a plurality of embedding vectors corresponding to reaction records stored in a database. The processor 950 may output a synthesis recipe corresponding to a query by retrieving a candidate reaction record corresponding to a candidate embedding vector in the database. The processor 950 may visualize synthesis recipes corresponding to a query by displaying the plurality of candidate embedding vectors together with the query embedding vector on an organic synthesis map, e.g., on the display 960.

The memory 930 may store a variety of information generated in the processing process of the processor 950 described above. Also, the memory 930 may store a variety of data and programs. The memory 930 may include a volatile memory or a non-volatile memory. The memory 930 may include a high-capacity storage medium such as a hard disk to store a variety of data.

Also, the processor 950 may perform at least one of the methods described above with reference to FIGS. 1 to 8 or an algorithm corresponding to at least one of the methods. The processor 950 may be a hardware-implemented data processing device having a circuit that is physically structured to execute desired operations. The desired operations may include, for example, code or instructions included in a program. The processor 950 may be implemented as, for example, a central processing unit (CPU), a graphics processing unit (GPU), or a neural network processing unit (NPU). For example, the prediction apparatus 900 that is implemented as hardware may include, for example, a microprocessor, a CPU, a processor core, a multi-core processor, a multiprocessor, an application-specific integrated circuit (ASIC), and a field programmable gate array (FPGA).

The processor 950 may execute a program and control the prediction apparatus 900. Program code to be executed by the processor 950 may be stored in the memory 930.

The display 960 may be any known display screen known to one of ordinary skill in the art such as a liquid crystal display (LCD) or an organic light-emitting diode (OLED).

The embodiments described herein may be implemented using a hardware component, a software component and/or a combination thereof. A processing device may be implemented using one or more general-purpose or special-purpose computers, such as, for example, a processor, a controller and an arithmetic logic unit (ALU), a digital signal processor (DSP), a microcomputer, an FPGA, a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is singular; however, one of ordinary skill in the art will appreciate that a processing device may include a plurality of processing elements and a plurality of types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be stored permanently or temporarily in any type of machine, component, physical or virtual equipment, computer storage medium, or device capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher-level code that may be executed by the computer using an interpreter.

The above-described hardware devices may be configured to act as one or more software modules in order to perform the operations of the above-described embodiments, or vice versa.

As described above, although the embodiments have been described with reference to the limited drawings, one of ordinary skill in the art may apply various technical modifications and variations based thereon. For example, suitable results may be achieved if the described techniques are performed in a different order, and/or if components in a described system, architecture, device, or circuit are combined in a different manner, and/or replaced or supplemented by other components or their equivalents. Accordingly, other implementations are within the scope of the following claims.

What is claimed is:

1. A method performed by at least one processor, the method comprising:
receiving a query comprising at least one of a product, a reactant, a reagent, and a reaction condition;
obtaining a query embedding vector corresponding to the query by inputting the query into a neural network model;
extracting a candidate embedding vector from among a plurality of embedding vectors based on a similarity between the query embedding vector and the plurality of embedding vectors corresponding to reaction records stored in a database; and
outputting a synthesis recipe corresponding to the query by retrieving a candidate reaction record corresponding to the candidate embedding vector in the database.

2. The method of claim 1, wherein the neural network model is trained to:
generate a target vector corresponding to the product for each synthesis recipe and a prediction vector corresponding to at least one of the reactant, the reagent, and the reaction condition, and
determine the synthesis recipe corresponding to the query or an embedding vector of the synthesis recipe corresponding to the query based on the target vector and the prediction vector.

3. The method of claim 1, wherein the extracting the candidate embedding vector comprises:
determining a similarity score based on a Euclidean distance between the query embedding vector and the plurality of embedding vectors; and
extracting the candidate embedding vector from among the plurality of embedding vectors based on the similarity score.

4. The method of claim 1, further comprising:
reducing a dimension of the query using principal component analysis,
wherein the inputting the query to the neural network model further comprises:
inputting the query of which the dimension is reduced to the neural network model to generate the query embedding vector.

5. The method of claim 1, wherein the product, the reactant, and the reagent have a molecular graph form that represents a three-dimensional (3D) molecular structure in a form of a graph.

6. The method of claim 1, further comprising:
generating a visual of one or more synthesis recipes corresponding to the query by displaying the candidate reaction record corresponding to the candidate embedding vector on an organic synthesis map.

7. The method of claim 1, further comprising:
generating, in a form of a graph, a 3D molecular structure to represent at least one element among the product, the reactant, and the reagent included in the query.

8. The method of claim 1, further comprising:
updating the neural network model based on user feedback on the synthesis recipe; and
inputting the query into the updated neural network model to generate an updated query embedding vector.

9. The method of claim 8, wherein the updating the neural network model further comprises:
storing an evaluation score corresponding to the user feedback in a query database;
updating the reaction records by reflecting the evaluation score in a similarity of an embedding vector corresponding to a corresponding synthesis recipe in the query database; and
updating the neural network model based on the updated reaction records.

10. A training method of a neural network model performed by at least one processor for determining a synthesis recipe, the training method comprising:
receiving a training query comprising at least one of a product, a reactant, a reagent and a reaction condition;
generating a target vector corresponding to the product for the synthesis recipe and a prediction vector corresponding to at least one of the reactant, the reagent, and the reaction condition by inputting the training query into the neural network model; and
training, based on the target vector and the prediction vector, the neural network model to determine the synthesis recipe corresponding to the training query or an embedding vector of the synthesis recipe corresponding to the training query.

11. The training method of claim 10, wherein the neural network model comprises:
at least one graph neural network comprising an encoder configured to extract a representation vector of a molecular unit corresponding to each of the product, the reactant, and the reagent;
a plurality of projection heads configured to convert the representation vector of the molecular unit corresponding to each of the product, the reactant, and the reagent to a low-dimensional latent vector; and
a feed-forward neural network configured to output a reaction vector corresponding to the reaction condition.

12. The training method of claim 11, wherein the plurality of projection heads comprise at least one of:
a first projection head configured to convert a first representation vector corresponding to the product to a first latent vector;
a second projection head configured to convert a second representation vector corresponding to the reactant to a second latent vector; and
a third projection head configured to convert a third representation vector corresponding to the reagent to a third latent vector.

13. The training method of claim 12, wherein the generating the target vector and the prediction vector further comprises:
applying the first representation vector to the first projection head to generate the first latent vector corresponding to the product to be the target vector;
applying the second representation vector to the second projection head to determine the second latent vector corresponding to the reactant;
applying the third representation vector to the third projection head to determine the third latent vector corresponding to the reagent;
applying the reaction condition to the feed-forward neural network to extract the reaction vector corresponding to the reaction condition; and
generating the prediction vector based on at least one of the second latent vector, the third latent vector, and the reaction vector.

14. The training method of claim 11, wherein the at least one graph neural network is configured to, in response to a missing element existing among the product, the reactant, and the reagent, provide a distribution value of a neighboring reaction record adjacent to a reaction record corresponding to the missing element in a database as a value corresponding to the missing element.

15. The training method of claim 11, wherein the plurality of projection heads are configured to, in response to a missing element existing among the product, the reactant, and the reagent input to the encoder, output a zero vector corresponding to the missing element.

16. The training method of claim 10, wherein the training the neural network model comprises training the neural network model by contrastive learning based on the target vector and the prediction vector.

17. The training method of claim 16, wherein the training the neural network model by contrastive learning comprises:
configuring a positive pair by the target vector and the prediction vector corresponding to a same synthesis recipe;
configuring a negative pair by the target vector and the prediction vector corresponding to different synthesis recipes; and
training the neural network model to learn a representation that causes the positive pair come closer to each other and the negative pair move away from each other in a vector space.

18. The training method of claim 10, further comprising:
receiving user feedback on the synthesis recipe generated by the neural network model;
updating a database based on additional training data generated by the user feedback; and
updating the neural network model based on the updated database.

19. A non-transitory computer-readable storage medium having instructions stored therein, which when executed by a processor cause the processor to execute a method comprising:
receiving a query comprising at least one of a product, a reactant, a reagent, and a reaction condition;
obtaining a query embedding vector corresponding to the query by inputting the query into a neural network model;
extracting a candidate embedding vector from among a plurality of embedding vectors based on a similarity between the query embedding vector and the plurality of embedding vectors corresponding to reaction records stored in a database; and
outputting a synthesis recipe corresponding to the query by retrieving a candidate reaction record corresponding to the candidate embedding vector in the database.

20. An apparatus comprising:
a communication interface configured to receive a query comprising at least one of a product, a reactant, a reagent, and a reaction condition;
a memory configured to store one or more instructions and a neural network model; and
a processor operatively coupled to the memory,
wherein the one or more instructions, when executed by the processor, cause the apparatus to:
obtain a query embedding vector corresponding to the query by inputting the query to the neural network model,
extract a candidate embedding vector from among a plurality of embedding vectors based on a similarity between the query embedding vector and the plurality of embedding vectors corresponding to reaction records stored in a database, and
output a synthesis recipe corresponding to the query by retrieving a candidate reaction record corresponding to the candidate embedding vector in the database.
